(19) United States
(12) Patent Application Publication
(10) Pub. No.: US 2006/0047167 A1
(43) Pub. Date: Mar. 2, 2006

(54) Method of synthesis of water soluble fullerene polyacids using a malonate reactant

(76) Inventors: Andreas Hirsch, Rathsberg (DE); Florian Beuerle, Erlangen (DE); Nikos Chronakis, Nicosia (CY)

Correspondence Address: WILLIAMS, MORGAN & AMERSON, 10333 RICHMOND, SUITE 1100, HOUSTON, TX, 77042, US

(21) Appl. No.: 11/214,468

(22) Filed: Aug. 29, 2005

Related U.S. Application Data
(60) Provisional application No. 60/606,780, filed on Sep. 2, 2004.
(60) Provisional application No. 60/668,230, filed on Apr. 4, 2005.

(51) Int. Cl. C07C 63/333; C07C 69/76

(52) U.S. Cl. 562/492; 560/82

(57) ABSTRACT

In one embodiment, the present invention is directed to a method for synthesizing compounds of the formula 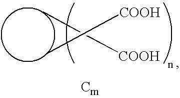
where Cₘ is a fullerene having m carbon atoms, the method comprising the steps of forming a linear malonate compound of the formula 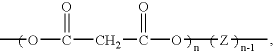
where each Z is the same or different and is a straight-chain or branched-chain aliphatic radical having from 1-30 carbon atoms which may be unsubstituted or monosubstituted or polysubstituted by identical or different substitutents, in which radicals up to every third —CH₂— unit can be replaced by O or NR″, n is an integer from 2 to 10, and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups; or (ii) a branched malonate compound of the formula 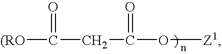
where Z′ is an aliphatic radical having from 1-30 carbon atoms which may be unsubstituted or monosubstituted or polysubstituted by identical or different substitutents, in which radicals up to every third —CH₂— unit can be replaced by O or NR″; and n is an integer from 2 to 10;

reacting said malonate compound with Cₘ to form an adduct of the formula 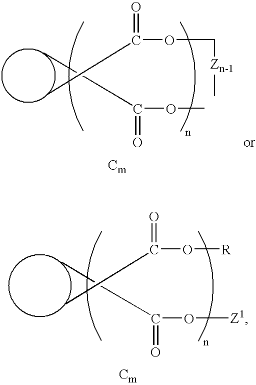 each Z is bonded to both one carboxylate group of a first malonate moiety and one carboxylate group of a second malonate moiety and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups; and hydrolyzing said adduct to form the compound.

This application claims priority from prior copending U.S. provisional patent applications Ser. No. 60/606,780, filed on Sep. 2, 2004, and Ser. No. 60/668,230, filed on Apr. 4, 2005.

### FIELD OF THE INVENTION

The invention relates generally to methods for the synthesis of substituted fullerene compounds and in particular to methods for the synthesis of carboxylated buckminsterfullerene (Cₘ) compounds, such as C₆₀ and C₇₀, among others. Even more specifically, the invention relates to methods for the synthesis of bis, tris and higher adducts of Cₘ.

### BACKGROUND OF THE INVENTION

Multiply-substituted fullerenes are useful for discovery of new pharmaceuticals. Murphy et al., U.S. Pat. No. 6,162,926, disclose multiply substituted fullerenes and describe their use in combinatorial libraries. The compounds have pharmaceutical, materials science and other utilities. FIGS. 1 and 2 are schematic representations of the bisadducts and trisadducts, respectively, disclosed in Murphy et al.

Using malonate groups (E¹—CH₂—E²) and the so-called Hirsch-Bingel reaction, fullerene compounds can be synthesized with groups substituted at many different sites. Wilson et al., Organic Chemistry of Fullerenes; Fullerenes: Chemistry, Physics and Technology, Kadish, K. M. and Ruoff, R. S., eds., John Wiley and Sons, New York, 2000, pp. 91-176.

Bingel, U.S. Pat. No. 5,739,376, describes the following reaction: 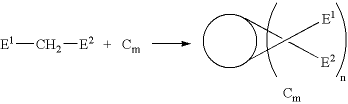
where E¹ and E² are COOH, COOR or other radicals, n is 1-10, and m is 60, 70, 76, or 78. Several of these compounds, e.g., the so-called carboxylated buckminsterfullerenes have become potentially useful as pharmaceutical candidates for the protection of neurotoxic injury. Choi and Dugan et al., PCT/EP97/02679. FIG. 3 depicts a trisadduct (C3) obtained by Choi.

The large scale synthesis of C3 is difficult since a multitude of isomers are produced and the preparation requires HPLC separation of the desired isomer for use as a therapeutic. One way to control the substitution of C₆₀ or other fullerene is by the so-called tether-directed addition process. Investigators have tried linking a multitude of chemically reactive groups together so that they react with the C₆₀ only at one site. A survey of these attempts is found in Wilson, et al.

It is known that water soluble fullerene hexaacids like those shown in FIGS. 4 and 5 are effective antioxidants and have neuroprotective properties. It is desirable to produce larger quantities of these compounds.

The usual synthetic precursors for the compounds of FIGS. 4 and 5 are the hexaesters. These can be made by stepwise reaction of a C₆₀ with diethylbromomalonate and intermediate purification by flash chromatography. The reaction has been described in Bingel., Chem. Ber. 1993, 126, 1957. Due to the stepwise synthesis and the tedious chromatographic purifications, the yield of trisadducts is low. This reaction is thus unsuitable for large scale production.

Diederich et al. have developed a method for the one-step production of e,e,e- and trans-3, trans-3, trans-3 trisadducts from C₆₀ using a cyclotriveratrylene tether. G. Rapenne et al., Chem. Commun. 1999, 1121. Although this reaction leads to a clean formulation of trisadducts, the overall yield is still quite low, e.g. 11% trans-3, trans-3, trans-3- and 9% e,e,e-isomer, and the tether system itself is only accessible in a multi-step synthesis.

While some success has been achieved using these methods to link one or more reactive groups to a single fullerene, more effective processes would be useful to prepare multiply substituted fullerenes for use in drug discovery or therapeutic applications.

### SUMMARY OF THE INVENTION

The invention is directed to methods for synthesizing compounds of the formula 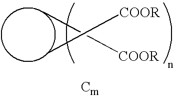
where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups.

The methods comprises one or more of the steps of forming a malonate compound; reacting said malonate compound with Cₘ to form an adduct; and hydrolyzing said adduct to form the compound.

### BRIEF DESCRIPTION OF THE DRAWINGS

FIG. 1 is a schematic representation of C₆₀ bis-adducts prepared by the methods of Murphy et al., U.S. Pat. No. 6,162,926.

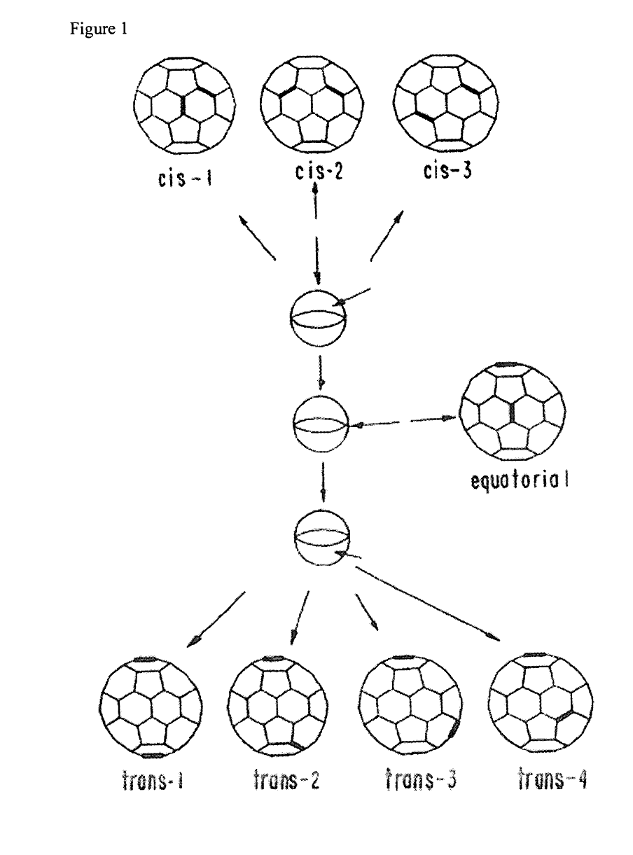

FIG. 2 is a schematic representation of C₆₀ tris-adducts prepared by the methods of Murphy et al., U.S. Pat. No. 6,162,926.

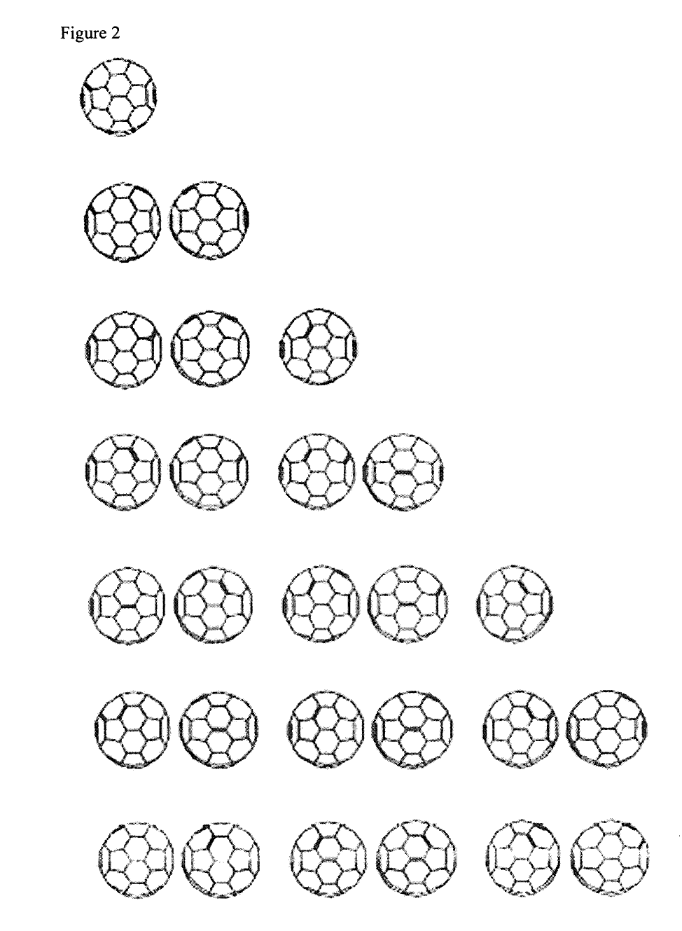

FIG. 3 is a schematic representation of the C₆₀ trisadduct prepared by the methods of Choi, International Application No. PCT/EP97/02679.

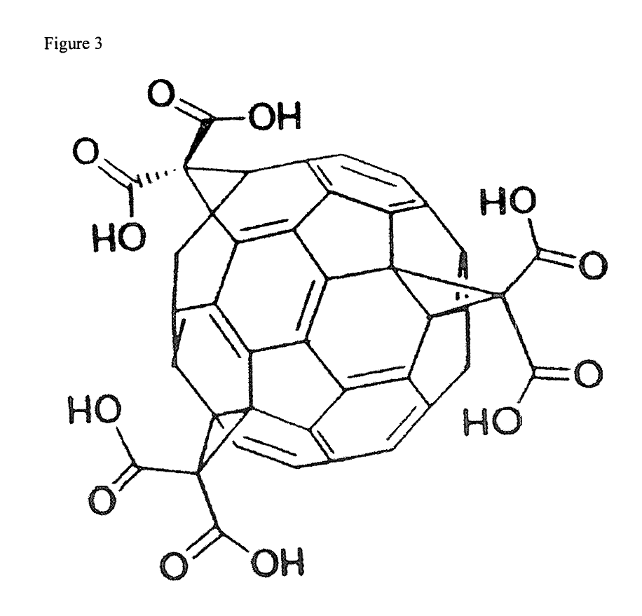

FIGS. 4 and 5 are schematic representations of the e,e,e-(1) and trans-3, trans-3, trans-3-(2) hexaacids of C₆₀.

FIG. 6 shows the synthesis scheme of the linear tris-malonate tether used in Example 1.

FIG. 7 shows an HPLC chromatogram of the reaction crude mixture of Example 1.

FIG. 8 shows the structure of the e,e,e-trisadduct E from the reaction of tether 4 with C₆₀, as described in Example 1.

FIG. 9 shows the synthesis scheme of a first branched tris-malonate tether.

FIG. 10 shows the synthesis scheme of a second branched tris-malonate tether.

FIG. 11 shows topologically distinct polar and equatorial fullerene addend zones and selective deprotection of the remote sites.

FIG. 12 shows the synthesis scheme of the tripodal tether molecule 5, as described in Example 3.

FIG. 13 shows the synthesis scheme of the tripodal tether molecule 10, as described in Example 3.

FIG. 14 shows the synthesis scheme of the tripodal tether molecule 12, as described in Example 3.

FIG. 15 shows the scheme for tether directed remote functionalization of C₆₀ with tripodal tris(malonate) tethers and subsequent selective deprotection of the addend zones.

FIG. 16 shows the NMR spectrum of the C₆₀ functionalized intermediate 13, as described in Example 3.

### DETAILED DESCRIPTION OF THE INVENTION

In one embodiment, the present invention is directed to a method for synthesizing compounds of the formula 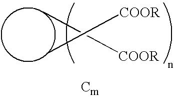
where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups. In one embodiment, m is 60 or 70. In another embodiment, each R is —(CH₂)₃—COOH.

The method comprises the steps of forming (i) a linear malonate compound of the formula 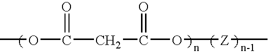
where each Z is the same or different and is a straight-chain or branched-chain aliphatic radical having from 1-30 carbon atoms which may be unsubstituted or monosubstituted or polysubstituted by identical or different substitutents, in which radicals up to every third —CH₂— unit can be replaced by O or NR″, n is an integer from 2 to 10, and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, or (ii) a branched malonate compound of the formula

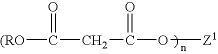

where Z′ is an aliphatic radical having from 1-30 carbon atoms which may be unsubstituted or monosubstituted or polysubstituted by identical or different substitutents, in which radicals up to every third —CH₂— unit can be replaced by O or NR″; and n is an integer from 2 to 10;

reacting said malonate compound with Cₘ to form an adduct of the formula

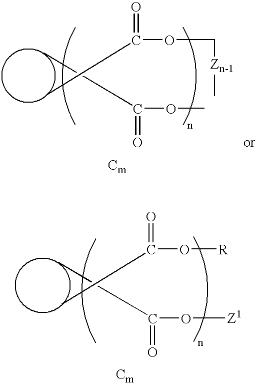

where each Z is bonded to both one carboxylate group of a first malonate moiety and one carboxylate group of a second malonate moiety and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups; and hydrolyzing said adduct to form a compound of the formula

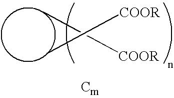

It has now been discovered that, in some embodiments, linking the malonate reactive groups in a linear molecule or a branched molecule and using the linear molecule or the branched molecule to react with Cₘ leads to improved yields of specific fullerene isomers and avoids the production of multiple undesirable addition isomers. It is possible, for example, to link two, three, four or five malonate groups in a linear molecule or a branched molecule.

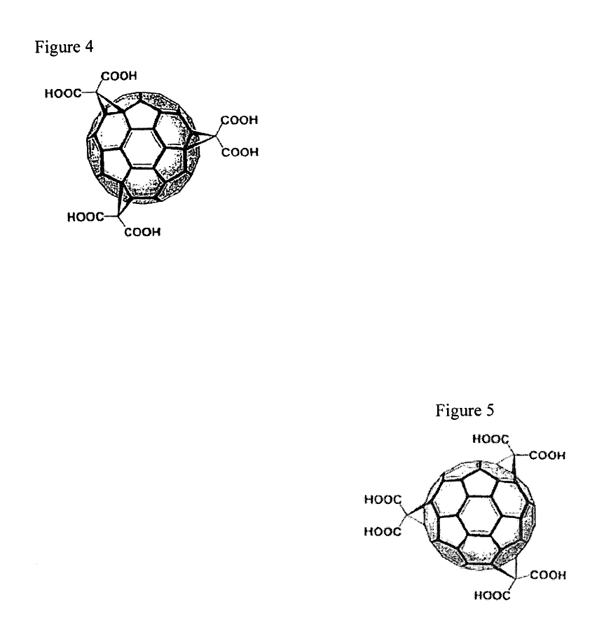

Linear molecules or branched molecules with three malonate units generally react cleanly with Cₘ to form trisadducts with high regioselectivity and in a typical reaction, an isolable yield of about 60 percent. The regiochemistry of the reaction can be “adjusted” between e,e,e and trans-3, trans-3, trans-3 by altering the chain length of the alkanediol or trialkanetridiol used to link the malonate reactive groups. Product purities of greater than 90 percent can be obtained by flash chromatography, the main impurity being the other regioisomers. The trisadducts thus obtained can be quantitatively hydrolyzed with sodium hydride to yield the water soluble hexaacids of FIGS. 4 and 5.

The linear malonate compound can be synthesized by reaction of a malonyl derivative, e.g. dichloride with a bifunctional moiety, e.g. a glycol. The bifunctional moiety may be termed a “Z precursor.” Methods to prepare such compounds are disclosed in Singh, J. Chem. Res. 1988, 132-133 and Singh, J. Chem. Res. 1989. In preferred embodiments, malonyl chloride is reacted with an alkanediol having from 8-18 carbon atoms. Octanediol is an exemplary Z precursor. A mixture of molecules of different lengths is obtained, and these can be separated by flash chromatography. The relative yields among these different molecules can be adjusted by altering the concentration of the reaction mixture.

The branched malonate compound can be synthesized by reaction of a malonyl derivative, e.g. malonyl chloride methyl ester, with a multifunctional moiety, e.g. a triol or triester in the case where n is 3. The trifunctional moiety may be termed a “Z′ precursor.” Methods to prepare such compounds are disclosed in Singh, J. Chem. Res. 1988, 132-133 and Singh, J. Chem. Res. 1989.

The compound of the formula

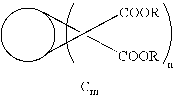

can undergo one or more of transesterification, deprotonation, or decarboxylation, among others reactions, to form esters, salts, lower acids, or other products which will be apparent to the skilled artisan having the benefit of the present disclosure, of the compound shown. Such reactions can be performed by the skilled artisan, or may happen spontaneously, depending on storage conditions (solvent, temperature, etc.).

In one embodiment of the invention the tetraacid of the formula

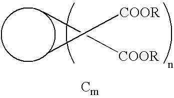

where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, wherein n=2, is formed by synthesizing a linear malonate compound of the formula 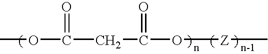
where Z is derived from octanediol and n=2; reacting said malonate compound with Cₘ to form the adduct of the formula

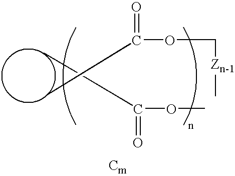

where each Z is bonded to both one carboxylate group of a first malonate moiety and one carboxylate group of a second malonate moiety, the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, and n=2; and hydrolyzing said adduct to form the tetraacid.

In a particular embodiment of the invention the hexaacid of the formula

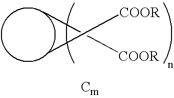

where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, wherein n=3, is formed by synthesizing a linear malonate compound of the formula

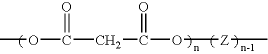

reacting said malonate compound with Cₘ to form the adduct of the formula

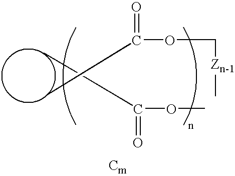

where each Z is bonded to both one carboxylate group of a first malonate moiety and one carboxylate group of a second malonate moiety, the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, and n=3,

and hydrolyzing said adduct to form the hexaacid.

In another particular embodiment of the invention, the hexaacid of the formula

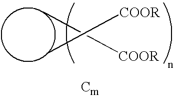

where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, wherein n=3, is formed by synthesizing a branched malonate compound of the formula

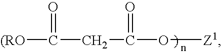

reacting said malonate compound with Cₘ to form the adduct of the formula

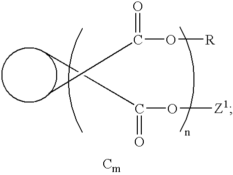

and hydrolyzing said adduct to form the hexaacid.

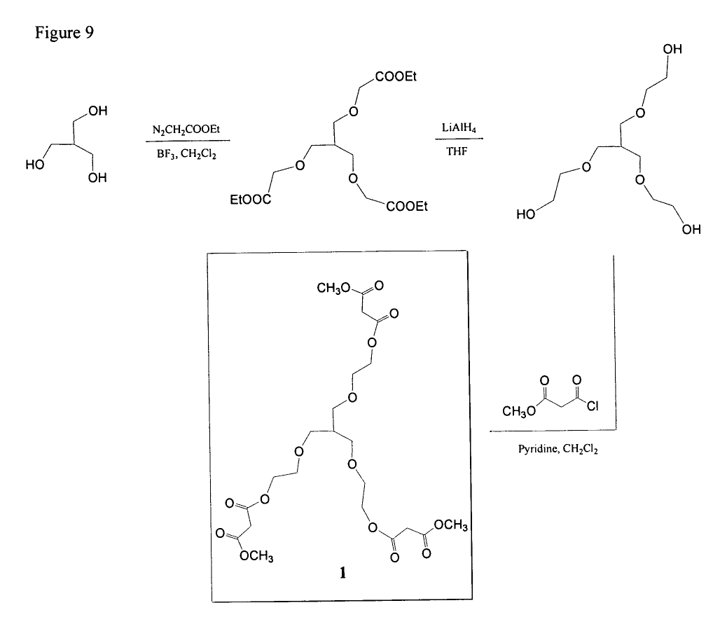

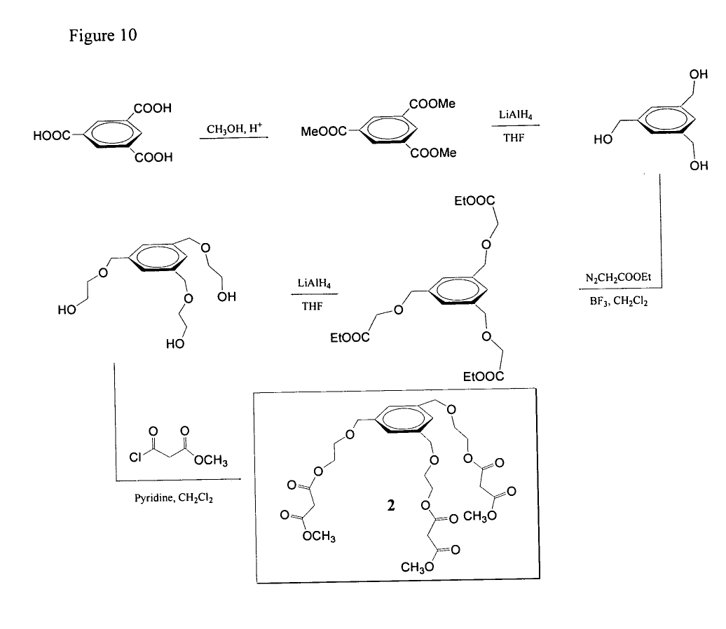

In one embodiment, the branched malonate compound can have the structure shown in FIG. 9 or FIG. 10 (i.e., each R is —CH₃ and Z is CH(CH₂O(CH₂)₂)—₃ or C₆(H)₃(CH₂O(CH₂)₂)—₃).

Other fullerene adducts can be synthesized using malonate compounds.

The cyclopropanation of the [60]fullerene cage via the Bingel reaction can theoretically lead to the formation of eight regioisomeric bis-adducts whereas, in the case of tris-adducts this number increases to 46. In 1994, we reported the synthesis and characterization of [60]fullerene tris-adducts via the stepwise nucleophilic cyclopropanation of the [6,6] bonds of the fullerene sphere. Tris-adducts with three-fold rotational symmetry like trans-3,trans-3,trans-3 and e,e,e were isolated, but this method required tedious chromatographic separations and purifications.

The concept of tethered systems connecting the reactive malonate groups has been proved a powerful tool to control the regioselectivity of tris-additions on C₆₀. In 1999, Diederich reported the regioselective synthesis of C₃-symmetrical tris-adducts by using a cyclotriveratrylene (CTV) tether connecting the malonate reactive groups. In this work, the all-trans-3 and all-e tris-adducts were isolated in 11% and 9% yields respectively, while the regioselective synthesis of C₆₀ tris-adducts with rotational symmetry in good yields was demonstrated in an elegant way by utilizing cyclo-[n]-alkylmalonate tethers with variable alkyl spacers connecting the malonate groups. Despite the improvements in the regioselective synthesis of C₆₀ tris-adducts, the tether approaches mentioned showed two disadvantages that should be taken into consideration. In contrast to the cyclo-[n]-alkylmalonates, the CTV tether required multiple synthetic steps whereas, in both cases the tethers do not offer the possibility of further structural tuning. Specifically, the tethered functionalized tris-adducts of C₆₀ can be only subjected to hydrolytic removal of the tether to afford the water soluble hexaacids of C₆₀. Further functionalization of the hexaacids was not possible due to decarboxylation phenomena.

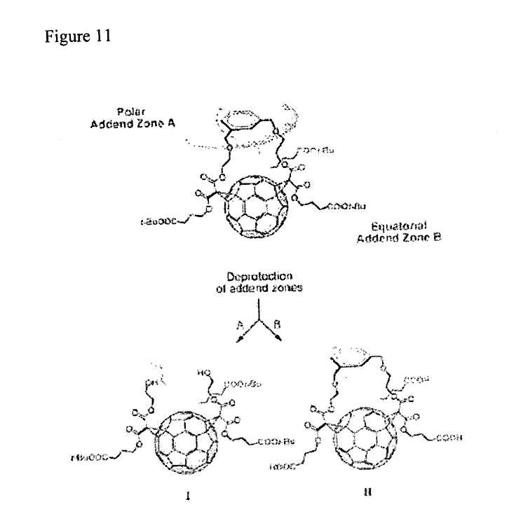

Our approach for the synthesis of derivatized [60]fullerene tris-adducts can allow: a) facile synthesis of tris(malonate) tethers, b) tunability of their structure by means of topologically distinct addend zones bearing protected functional groups, and c) subsequent selective chemical transformations i.e., deprotection of the functional groups. For this purpose, we have developed the synthesis of tripodal tris(malonate) tethers where, the malonate reactive groups are connected via alkyl spacers with a benzene core, described as the focal point of the tether. The second ester moiety of each malonate is terminated by another protecting group. The concept of the newly designed tethers is demonstrated in FIG. 11. The tris-adducts derived from the Bingel cyclopropanation of C₆₀ with this family of tethers possess two distinct addend zones namely, polar zone A and equatorial zone B. Zone A represents the focal point of the tether where the hydroxyl terminal groups of the alkyl spacers located in the pole of C₆₀ are connected/protected with a benzene core. Zone B includes the tertbutyl ester functional groups terminating the alkyl substituents of the malonic ester moieties around the equator of C₆₀. The selective deprotection of the addends in zone A or B is expected to provide facile access to the direct synthesis of the C₆₀ tris-adducts I and II, respectively. As was mentioned before, these structurally novel trisadducts are not accessible starting from the e, e, e tris(malonic acid) of C₆₀. Finally, the fullerene cage can also be regarded as a reactive zone, taking into account the possibility of further functionalization in targeting hexa-adducts, as well as the fact that, to a certain extent, it retains the unique electronic properties of a fullerene molecule.

In the following Example, the hexaacid of FIG. 4 is obtained by the reaction of Cₘ with a linear malonate compound and subsequent hydrolysis. Other reactions follow the same principle.

### EXAMPLE 1

SYNTHESIS OF THE e,e,e-TRISADDUCT OF [60]FULLERENE UTILIZING THE TETHER-DIRECTED REMOTE FUNCTIONALIZATION WITH AN OPEN TRIS-MALONATE TETHER

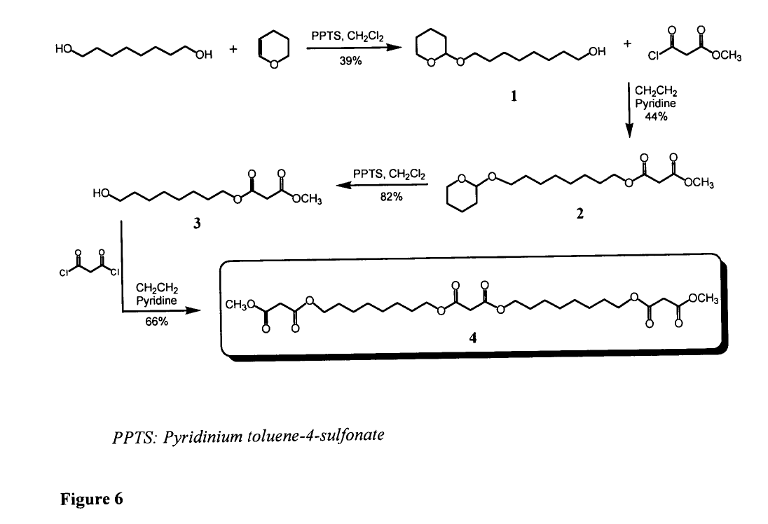

A new series of tris-malonate tethers that possess an open structure and bear alkyl groups as spacers were tested. The synthesis of one such tether that worked well with C₆₀ is described in FIG. 6. PPTS: Pyridinium toluene-4-sulfonate.

The intermediates of the synthesis of FIG. 6 were purified by flash column chromatography as follows, and were fully characterized by ¹³C-, ¹H-NMR and mass spectrometry (FAB-MS).

1: SiO₂, Hexane/EtOAc=1/1, Colorless oil.

2: SiO₂, Hexane/EtOAc=7/3, Colorless oil.

3: SiO₂, Hexane/EtOAc=3/2, Colorless oil.

4: SiO₂, Hexane/EtOAc=1/1, Colorless oil.

The yield of the first step of the synthesis was 39% because the bis-protected diol was also formed. The reason is that 1,8-octane-diol was not well soluble in CH₂Cl₂ and consequently, the mono-protected diol (soluble in CH₂Cl₂) was subjected to a rapid second reaction. We expect the use of larger amounts of solvent will improve the yield of the first step of the synthesis. Yields about 85% have been reported for similar diols according to the experimental procedure we followed. For details see: H. M. S. Kumar, B. V. S. Reddy, E. J. Reddy, J. S. Yadav, Chemistry Letters, 1999, 857-858.

The reaction of C₆₀ with tether 4 was performed under known experimental conditions, already reported for the modified Bingel addition. In a typical procedure, 101 mg of C₆₀ (0.14 mmoles) were dissolved in 160 ml of dry toluene under a nitrogen atmosphere. Subsequently, 73 mg of the tether 4 (0.13 mmoles) and 100 mg of I₂ (0.39 mmoles) were added, followed by the dropwise addition of a solution of 1,8-diazabicyclo-[5.4.0]undec-7-ene (DBU) (145 μl, 0.97 mmoles) in 60 ml of dry toluene over a period of 2 hours. The reaction mixture was stirred at room temperature for 21 hours, filtered through a paper filter, and subjected to flash column chromatography on silica gel (6×25 cm). Traces of unreacted C₆₀ and other impurities were eluted with toluene, then the eluent was changed to toluene/ethylacetate=98/2 and the trisadducts were eluted.

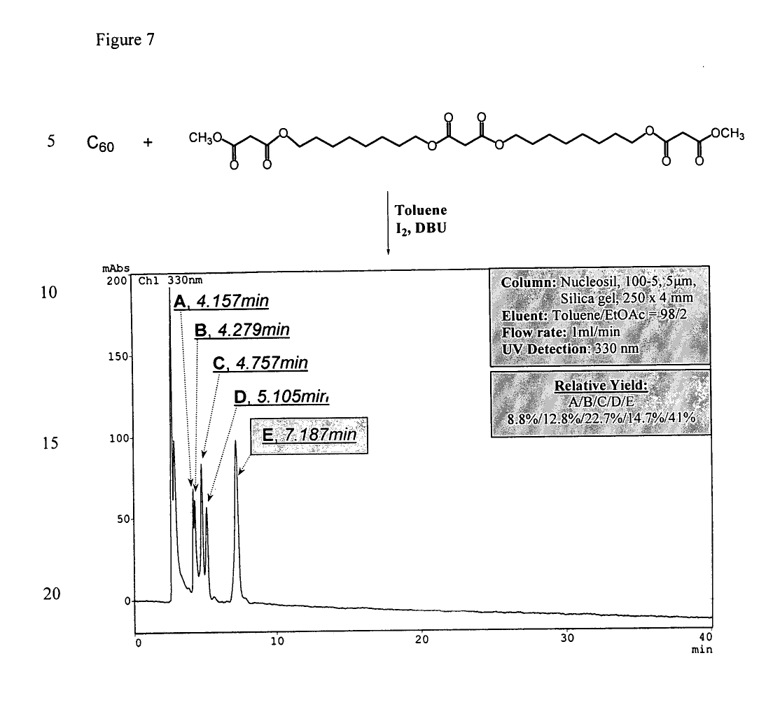

In FIG. 7, the HPLC chromatogram of the crude reaction mixture is shown. It can be observed that five different trisadducts (A, B, C, D, E) were formed during the reaction. The expected molecular ion was observed in the FAB-MS, thus, proving the formation of adducts derived from the succesive three-fold Bingel addition of tether 4 to C₆₀. Trisadduct E was the major product and the relative yield calculated from the HPLC was 41%. It was the trisadduct with the larger retension time (7.187 min) and was eluted last from the column. It was isolated in pure form by flash columm chromatography on SiO₂ with toluene/ethylacetate=98/2 as eluent, as it was the last of the trisadducts eluted. It was eluted as a cherry-red coloured band. 44 mg were isolated as a cherry-red solid (yield: 26%). The solubility in solvents like CHCl₃ or CH₂Cl₂ was excellent.

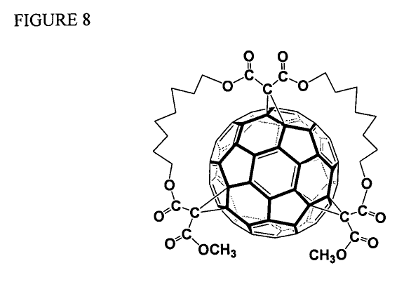

Trisadduct E was characterized by UV, FAB-MS, ¹³C- and ¹H-NMR spectroscopic methods and its addition pattern was assigned as the e,e,e (FIG. 8).

### EXAMPLE 2

Branched malonate molecules 1 and 2 were produced according to the schemes shown in FIGS. 9-10.

### EXAMPLE 3

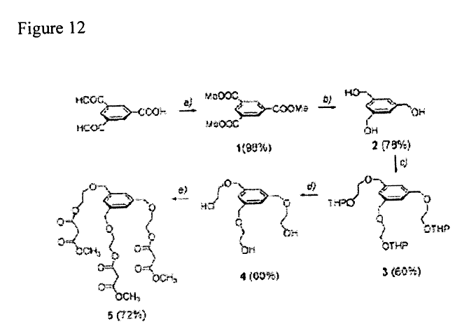

The synthesis of a tripodal trismalonate tether (5) is shown in FIG. 12. Triol 4 was synthesized starting from benzene-1,3,5-tricarboxylic acid according to a literature procedure. Treatment of 4 with methyl 3-chloro-3-oxopropionate in the presence of pyridine in CH₂Cl₂, followed by flash column chromatographic purification, afforded pure 5 in 72% yield.

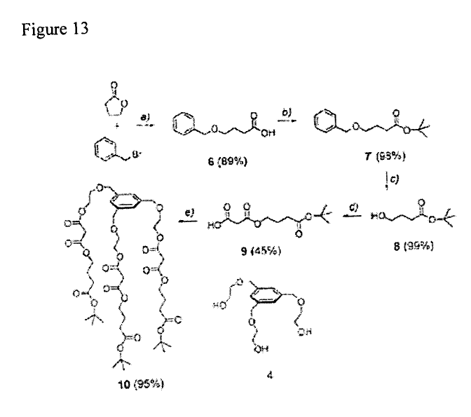

Targeting tripodal tethers bearing easily removable protective groups in the side chains, we performed the synthesis of tether 10 (FIG. 13), where the malonic ester moieties are further elongated with C₃ alkyl chains terminated by tert-butyl ester groups. In this case, selective hydrolysis of the ester moieties or focal deprotection (debenzylation) of the formed tris-adducts of C₆₀ can give a facile access to structurally different derivatives. For this purpose, tert-butyl 4-hydroxybutyrate (8) was prepared (FIG. 13) and then subjected to a DCC monoesterification reaction with malonic acid to yield the mono-protected diacid 9. Three-fold esterification of trio 4 with acid 9 by using DCC and DMAP in CH₂Cl₂, afforded the tether 10 in 95% isolated yield.

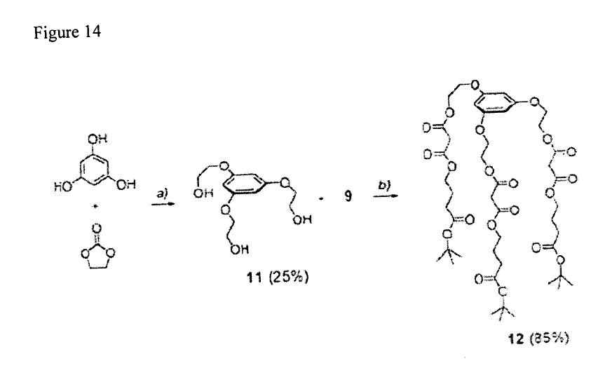

Molecular modelling studies showed that replacement of the focal benzyl site by a phenyl group favors the regioselective formation of the e, e, e fullerene tris-adduct. It is postulated that the reduction of the tether length is responsible for the increased calculated thermodynamic stability of the e,e,e regioisomer over other isomers such as, for example, the trans-3,trans-3,trans-3. Consequently, tether 10 was modified by replacing the benzyloxy protective group with phenoxy, thus shortening each spacer by one carbon atom. For this purpose, triol 11 was synthesized in one step followed by a DCC esterification reaction with acid 9 (FIG. 14). The reaction was performed in THF, as 11 was insoluble in CH₂Cl₂, and, after chromatographic purification, tether 12 was obtained in pure form in 85% yield.

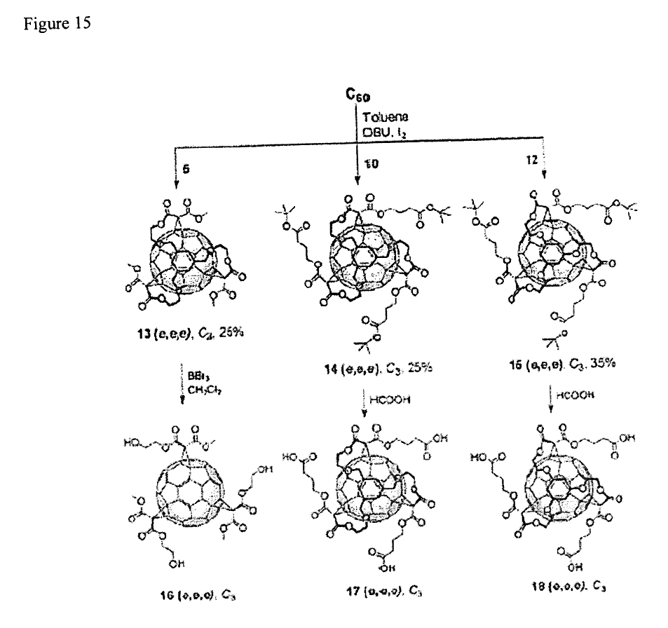

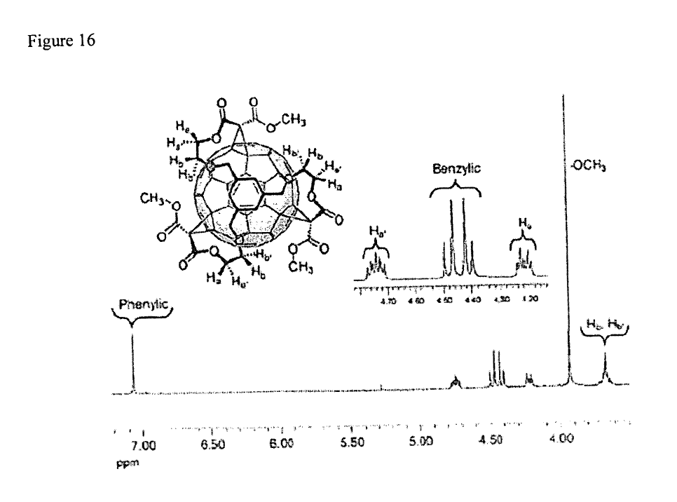

We then investigated the Bingel functionalization of C₆₀ with the D₃ₕ-symmetrical tether 5. The reaction was carried out at a concentration of 0.55 mmol L⁻¹ of C₆₀ in toluene, in the presence of I₂ and 1,8-diazabicyclo[5.4.0]undec-7-ene (DBU). Flash column chromatographic separation of the crude mixture (SiO₂, toluene-EtOAc, 70:30) afforded two fractions, which were further analyzed by FAB-MS and HPLC. The first, least polar fraction, showed the expected 1315 m/z molecular ion in the FAB-MS spectrum, thus confirming that the three-fold Bingel cyclopropanation occurred successfully on C60. The HPLC elugram consisted of one peak but ¹H and ¹³C NMR analysis revealed that this fraction was a mixture of tris-adducts, not separable by chromatographic methods. The second, most polar fraction consisted of a single tris-adduct and was formed in 55% relative yield. The structure of 13 was assigned by comparison of its UV/V is spectra with those of previously reported e,e,e tris-adducts. The ¹H and ¹³C NMR spectroscopic data were in agreement with an e,e,e addition pattern (FIG. 15). In the fullerene spectral region between 140 and 148 ppm, 17 of the 18 expected signals for the sp² carbon atoms of the fullerene are observed, indicating a C₃ symmetry. The signal at 146.64 ppm is of double intensity. In addition, two signals for the fullerene sp³ carbons at 69.72 and 70.65 ppm, and one signal for the bridgehead sp³ C-atoms at 52.56 ppm are present in the spectrum while, the carbonyl C-atoms show two absorptions at 163.20 and 163.82 ppm. The ¹H NMR spectra (FIG. 16) shows a singlet absorption at 7.08 ppm for the phenylic protons and two doublets at 4.43 and 4.50 ppm for the diastereotopic benzylic hydrogens, while it is worth noting that the two diastereotopic methylenic protons Hₐ and Hₐ experience totally different chemical environments reflected in the large difference between their chemical shifts (0.53 ppm). These protons resonate at 4.23 and 4.76 ppm, correspondingly. Tris-adduct 13 was isolated in pure form (SiO₂, toluene-EtOAc, 70:30) as a cherry-red solid, in 25% yield.

The Bingel cyclopropanation of C₆₀ with the D₃ₕ-symmetrical tether 10 was carried out under the same experimental conditions used in the reaction of C₆₀ with tether 5. Tether 10 showed similar regioselectivity, leading to the formation of a mixture of nonseparable tris-adducts eluted in a single fraction (SiO₂, toluene-EtOAc, 70:30) and the e,e,e regioisomer 14, which was formed in 55% relative yield (FIG. 15). Tris-adduct 14 was isolated in 25% yield and characterized by ¹H, ¹³C NMR and UViVis spectroscopy, and FAB-MS.

An improved enhancement in the regioselectivity of the Bingel tris-addition was observed when C₆₀ was treated with the tripodal tether 12 in toluene, in the presence of I₂ and DBU. The reaction afforded with complete regioselectivity the C₃-symmetrical e,e,e tris-adduct 15 (FIG. 15) which was purified by flash column chromatography on SiO₂ using a mixture of toluene-EtOAc, 80:20, as eluent. The addition pattern was unambiguously assigned by ¹H, ¹³C NMR, and UV/vis spectroscopy and 15 was isolated in pure form in 35% yield.

With the successfully synthesized and characterized e, e, e trisadducts 13, 14, and 15 in hand, we attempted in the next step the selective deprotection of the distinct addend zones. The deprotection of the benzyloxy moiety of tris-adduct 13 (focal deprotection) was carried out in the present of a Lewis acid as it had been reported that removal of the O-benzyl groups of sugar fullerene derivatives by palladium catalyzed hydrogenolysis, afforded a complex mixture due to decomposition of C₆₀. A rapid reaction was observed on the addition of BBr₃ to a solution of 13 in CH₂Cl₂ at −70° C., and the formed e, e, e triol 16 (FIG. 15) was isolated by flash column chromatography (SiO₂, CH₂Cl₂—CH₃OH, 95:5). The FAB-MS showed the expected M⁺ molecular ion at m/z 1201, whereas the UV/Vis spectrum was in full agreement with the e, e, e addition pattern. Furthermore, the treatment of tris-adducts 14, 15 with formic acid led to the hydrolysis of the tert-butyl ester groups to form the corresponding tris-acids 17 and 18 respectively, as demonstrated by FAB-MS and UV/Vis spectroscopy.

In conclusion, we have synthesized a new family of tripodal D₃ₕ-symmetrical tris(malonate) tethers and investigated their regioselectivity in the Bingel cyclopropanation of C₆₀. Tuning of the spacer length allows for a significant improvement in selectivity for e,e,e regioisomer formation whereas selective deprotection of the topologically distinct polar and equatorial addend zones provides facile synthetic access to appealing building blocks for further selective functionalization.

What is claimed is:

1. A method for synthesizing compounds of the formula 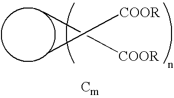
where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, comprising the steps of:
(a) forming (i) a linear malonate compound of the formula 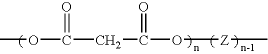
where each Z is the same or different and is a straight-chain or branched-chain aliphatic radical having from 1-30 carbon atoms which may be unsubstituted or monosubstituted or polysubstituted by identical or different substitutents, in which radicals up to every third —CH₂— unit can be replaced by O or NR″, n is an integer from 2 to 10, and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, or (ii) a branched malonate compound of the formula 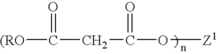
where Z′ is an aliphatic radical having from 1-30 carbon atoms which may be unsubstituted or monosubstituted or polysubstituted by identical or different substitutents, in which radicals up to every third —CH₂— unit can be replaced by O or NR″; and n is an integer from 2 to 10;
(b) reacting said linear malonate compound or said branched malonate compound with Cₘ to form an adduct of the formula 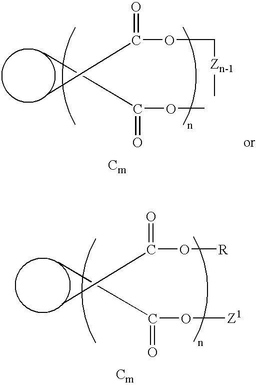
where each Z is bonded to both one carboxylate group of a first malonate moiety and one carboxylate group of a second malonate moiety and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, and
(c) hydrolyzing said adduct to form the compound.

2. A method as recited in claim 1 wherein n is 2.

3. A method as recited in claim 1 wherein n is 3.

4. A method as recited in claim 1 wherein Z is derived from a diol.

5. A method as recited in claim 1 wherein each Z is derived from an alkanediol containing 8-10 carbon atoms.

6. A method as recited in claim 1 wherein Z is derived from octanediol.

7. A method as recited in claim 1, wherein each R is —CH₂)₃—COOH.

8. A method for synthesizing compounds of the formula 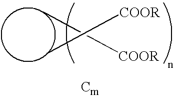
where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, comprising the steps of:
(a) reacting (i) a linear malonate compound of the formula 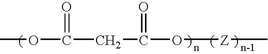
where each Z is the same or different and is a straight-chain or branched-chain aliphatic radical having from 1-30 carbon atoms which may be unsubstituted or monosubstituted or polysubstituted by identical or different substitutents, in which radicals up to every third —CH₂— unit can be replaced by O or NR″, n is an integer from 2 to 10, and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, or (ii) a branched malonate compound of the formula 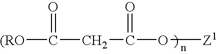
where Z′ is an aliphatic radical having from 1-30 carbon atoms which may be unsubstituted or monosubstituted or polysubstituted by identical or different substitutents, in which radicals up to every third —CH₂— unit can be replaced by O or NR″; and n is an integer from 2 to 10;
with Cₘ to form an adduct of the formula 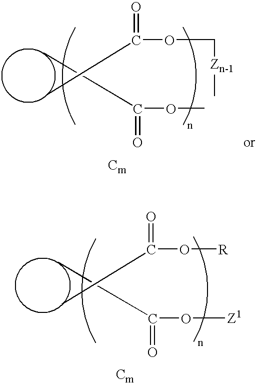
where each Z is bonded to both one carboxylate group of a first malonate moiety and one carboxylate group of a second malonate moiety and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, and
(b) hydrolyzing said adduct to form the compound.

9. A method as recited in claim 8 wherein n is 2.

10. A method as recited in claim 8 wherein n is 3.

11. A method as recited in claim 8 wherein Z is derived from a diol.

12. A method as recited in claim 8 wherein each Z is derived from an alkanediol containing 8-10 carbon atoms.

13. A method as recited in claim 8 wherein Z is derived from octanediol.

14. A method as recited in claim 8, wherein each R is —CH₂)₃—COOH.

15. A method for synthesizing compounds of the formula 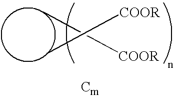
where Cₘ is a fullerene having m carbon atoms and each R is independently —H or —R″—COOH where R″ is alkyl having 1-20 carbon atoms, substituted alkyl having 1-20 carbon atoms, alkenyl having 1-20 carbon atoms, substituted alkenyl having 1-20 carbon atoms, or a chain containing unsubstituted or substituted aryl or other cyclic groups, comprising the steps of hydrolyzing an adduct of the formula 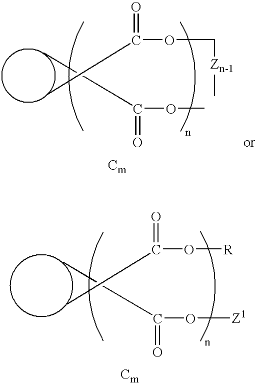
where each Z is bonded to both one carboxylate group of a first malonate moiety and one carboxylate group of a second malonate moiety and the unfilled valences of the first and nth malonate groups are filled with bonds to hydrogen, a hydrocarbon group or substituted hydrocarbon group having 1-20 carbon atoms, or a chain containing unsubstituted or substituted aryl or other cyclic groups, to form the compound.

16. A method as recited in claim 15 wherein n is 2.

17. A method as recited in claim 15 wherein n is 3.

18. A method as recited in claim 15 wherein Z is derived from a diol.

19. A method as recited in claim 15 wherein each Z is derived from an alkanediol containing 8-10 carbon atoms.

20. A method as recited in claim 15 wherein Z is derived from octanediol.

21. A method as recited in claim 15, wherein each R is —CH₂)₃—COOH.
